(19) United States
(12) Patent Application Publication
(10) Pub. No.: US 2023/0277814 A1
(43) Pub. Date: Sep. 7, 2023

(54) Removable Skin Nicking Blade for Catheter Placement System

(71) Applicant: Bard Access Systems, Inc., Salt Lake City, UT (US)

(72) Inventor: Glade H. Howell, Draper, UT (US)

(21) Appl. No.: 18/117,334

(22) Filed: Mar. 3, 2023

Related U.S. Application Data
(60) Provisional application No. 63/316,271, filed on Mar. 3, 2022.

(51) Int. Cl. A61M 25/06; A61M 25/00; A61M 25/01; A61M 25/09

(52) U.S. Cl. CPC A61M 25/0612; A61M 25/0026; A61M 25/0136; A61M 25/0606; A61M 25/09041; A61M 2025/0183; A61M 2025/0024

(57) ABSTRACT

A catheter placement system including a removable skin nicking device coupled with a catheter, e.g., a Rapidly Insertable Central Catheter. The skin nicking device includes a handle and a blade extending therefrom. The blade is disposed within a notch extending along a dilation section of the catheter. The blade includes a radially outward directed sharp edge configured to cut the skin adjacent a catheter insertion site. A distal end of the blade is disposed within the notch to prevent a skin bridge during insertion of the catheter. A needle is inserted into an access lumen of the catheter via a needle access port and an access guidewire is disposed within a lumen of the needle. An advancement guidewire is also disposed within the access lumen.

### PRIORITY

This application claims the benefit of priority to U.S. Provisional Application No. 63/316,271, filed Mar. 3, 2022, which is incorporated by reference in its entirety into this application.

### BACKGROUND

Central venous catheters (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma or increased risk of infection. There is a relatively high potential for touch contamination due to the number of medical devices that need to be interchanged during the Seldinger technique. As such, rapidly insertable central catheter (RICC) placement systems have been developed to reduce the number of steps and medical devices involved in placing a catheter, such as a CVC, into a patient.

Whether using a traditional Seldinger techniques or using catheter placement systems, often the clinician is required widen an insertion site after the vasculature has been accessed. This involves sliding a rear side, or dull side, of a scalpel along a guidewire that is stabilizing the insertion site. The sharp side of the scalpel can be facing outwards. As the scalpel is slid over the guidewire into the insertion site, the sharp edge can engage and widen the insertion site in preparation for the CVC to be advanced therethrough.

However, great skill is required to maintain contact between the scalpel tip and the guidewire, as the scalpel is slid therealong. The scalpel must be aligned perfectly with the guidewire and held firmly in place. Gaps between the scalpel and the guidewire can result in “skin bridges” being formed across the insertion site, complicating the procedure. Embodiments described herein are directed to address the foregoing.

### SUMMARY

Disclosed herein is a catheter placement system, that according to some embodiments, includes a catheter comprising including a dilation section, an access section extending distally away from the dilation section, a catheter section extending proximally away the dilation section, and a notch extending longitudinally along at least a portion of the dilation section. The system further includes a skin nicking device coupled with the catheter, where the skin nicking device includes a handle and a blade extending distally away from the handle, and where a portion of the blade is disposed within the notch. In some embodiments, the blade is removable from the notch.

In some embodiments, the catheter is a rapidly insertable central catheter and, in some embodiments, the access section is mono-luminal and the catheter section is multi-luminal.

In some embodiments, the access section defines a first outer diameter, the catheter section defines a second outer diameter greater than the first outer diameter, and the dilation section defines a tapered outer profile that includes the first outer diameter at a distal end of the dilation section and the second diameter at a proximal end of the dilation section.

In some embodiments, the notch defines a width that is less than a thickness of the blade such that the notch defines an interference fit with the blade. In some embodiments, 7 a dull edge of the blade is disposed within the notch and a sharp edge of the blade is directed radially outward from the catheter. In some embodiments, the sharp edge defines an angle with respect to a longitudinal axis of the catheter such that a distal portion of the sharp edge is closer to the longitudinal axis than a proximal portion of the sharp edge. In some embodiments, the sharp edge is parallel with an outer surface of the dilation section.

In some embodiments, a distal end of the blade is disposed radially inward of the outer surface of the dilation section. In some embodiments, the blade includes a tab extending distally from the distal end of the blade, the notch includes a cavity at a distal end of the notch, and the tap is disposed within the cavity. In some embodiments, during use, a distally oriented force applied to the handle by a clinician is transferred to the catheter via the cavity. In some embodiments, the handle extends proximally along the catheter section. In some embodiments, the notch is configured to align the blade with the longitudinal axis.

In some embodiments, the notch is formed of a slit such that upon removal of the blade from the notch, the notch transitions to a closed state, where the outer surface of the dilation section forms a continuous circumferential surface.

In some embodiments, the catheter includes an access lumen extending along the access, dilation, and catheter sections, and the access section includes a needle access port in fluid communication with the access lumen.

In some embodiments, the system further includes a needle disposed within the access lumen between the needle access port and a distal end of the catheter.

In some embodiments, the system further includes an access guidewire disposed within a lumen of the needle. In some embodiments, the system further includes an advancement guidewire disposed within the access lumen proximal access port.

Also disclosed herein is a method of placing a catheter within a vasculature of a patient that, according to some embodiments, includes (i) forming an insertion site with a needle disposed within an access lumen of an access section of catheter, where the needle is inserted through a needle access port of the access section; (ii) advancing a dilation section disposed proximal the access section into the insertion site, where the dilation section is coupled with a skin nicking device having a blade; (iii) cutting a skin adjacent the insertion site with a sharp edge of the blade during advancement of the dilation section; (iv) dilating the insertion site from a first diameter of the access section to a second diameter of a catheter section disposed proximal the dilation section during advancement of the dilation section; (v) separating the skin nicking device from the dilation section; and (vi) advancing the catheter section through the insertion site into the vasculature.

In some embodiments, the blade includes a handle, and advancing a dilation section includes applying a distally oriented force to the handle.

In some embodiments, the method further includes advancing an access guidewire through a lumen of the needle and into the vasculature, and in some embodiments, the method further includes advancing an advancement guidewire through the access lumen and into the vasculature.

In some embodiments, a portion of the blade is disposed within a notch extending along a least a portion of the dilation section, where the notch includes a cavity at a distal end thereof, and the blade includes a tab extending distally away from a distal end of the blade. In such embodiments, the tab is disposed within the cavity such that the distally oriented force applied to the handle is transferred to the catheter via the tab disposed within the cavity.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a plan view of a rapidly insertable central catheter (catheter) placement system including a removable skin nicking device, in accordance with embodiments disclosed herein;

FIG. 2 shows close up detail of the removable skin nicking device coupled with a distal portion of a catheter, in accordance with embodiments disclosed herein;

FIG. 3A shows a lateral cross section of the removable skin nicking device engaged with the catheter, in accordance with embodiments disclosed herein;

FIG. 3B shows a lateral cross section of the removable skin nicking device disengaged from the catheter, in accordance with embodiments disclosed herein;

FIG. 4 shows a longitudinal cross section of the removable skin nicking device engaged with the catheter, in accordance with embodiments disclosed herein;

FIGS. 5A-5E illustrate various views of the system 100 show an exemplary method of use for a catheter placement system including a removable skin nicking device, in accordance with embodiments disclosed herein; and

FIG. 6 illustrates a block diagram of a method of placing the catheter of FIG. 1 within a vasculature of a patient, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

Unless defined otherwise, all technical and scientific terms used herein have the same meaning as commonly understood by those of ordinary skill in the art.

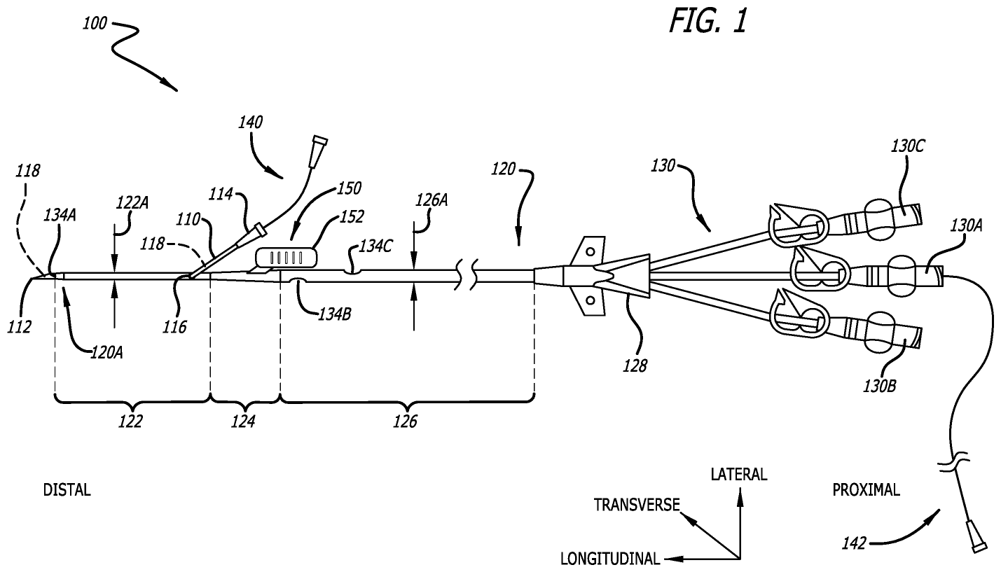

To assist in the description of embodiments described herein, as shown in FIG. 1, a longitudinal direction extends substantially parallel to an axial length of the catheter. A lateral direction extends normal to the longitudinal direction, and a transverse direction extends normal to both the longitudinal and lateral axes.

FIG. 1 shows an exemplary catheter placement system (“system”) 100, generally including a needle 110, a catheter 120 (e.g., a Rapidly Insertable Central Catheter), an access guidewire 140, and an advancement guidewire 142. In some embodiments, the system 100 may include additional guidewires. The needle 110 may include a needle hub 114 disposed at a proximal end thereof. In some embodiments, the system 100 can further include a blood flash system (not shown) configured to draw a fluid from a distal tip 112 of the needle 110 to confirm that the distal tip 112 of the needle is correctly placed within vasculature.

The catheter 120 includes an access section 122 extending proximally away from a distal end 120A of the catheter 120, a catheter section 126 extending distally away from a catheter hub 128, and a dilation section 124 disposed between the access section 122 and the catheter section 126. The access section 122 can define a single lumen and a first outer diameter 122A. The catheter section 126 can define more than one lumen and a second outer diameter 126A, where the second outer diameter 126A is greater than the first outer diameter 122A. In the illustrated embodiment, the catheter section 126 defines three lumens. However, it will be appreciated that the catheter section 126 may define more or less than three lumens. The dilation section 124 can define a tapered outer profile having the first outer diameter 122A at a distal end of the dilation section 124 and the second outer diameter 126A at a proximal end of the dilation section 124.

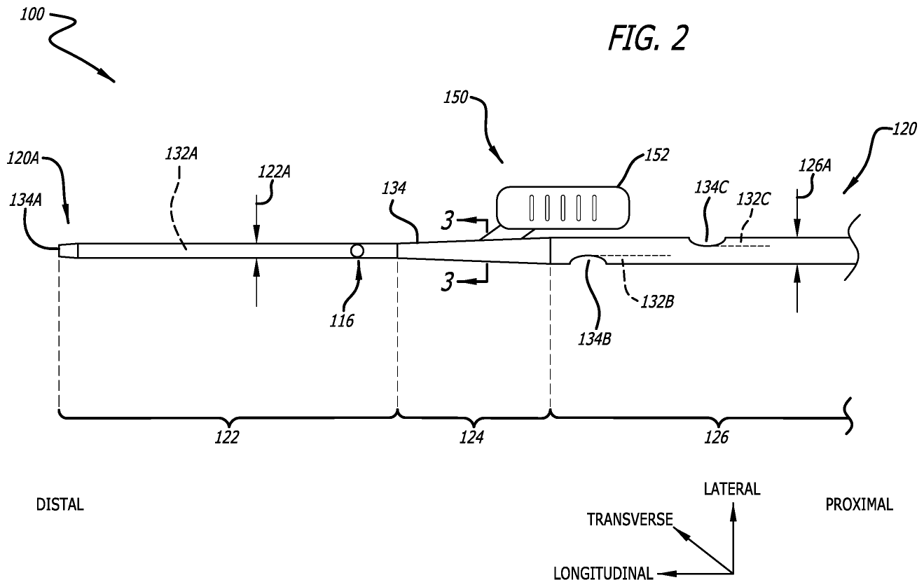

The hub 128 is disposed at a proximal end of the catheter section 126 and is coupled with one or more extension legs 130 extending proximally therefrom. Each extension leg 130 can be in fluid communication with a lumen of the catheter 120. For example, a first extension leg 130A can be in fluid communication with a distal (or access) lumen 132A (see FIG. 2), a second extension leg 130B can be in fluid communication with a medial lumen 132B, and a third extension leg 130C can be in fluid communication with a proximal lumen 132C. The distal lumen 132A can extend to a distal lumen opening 134A disposed at the distal end 120A of the catheter 120. The medial lumen 132B can extend to a medial lumen opening 134B extending through a wall of the catheter section 126. The proximal lumen 132C can extend to a proximal lumen opening 134C extending through a wall of the catheter section 126.

In an embodiment, the medial lumen opening 134B can be disposed adjacent the dilation section 124 and the proximal lumen opening 134C can be disposed proximal of the medial lumen opening 134B or vice versa. In an embodiment, the medial lumen opening 134B and the proximal lumen opening 134C can be disposed equidistant from the distal lumen opening 134A. In an embodiment, one or both of the medial lumen opening 134B and the proximal lumen opening 134C can be disposed along the dilation section 124.

In an embodiment, the needle 110 can extend through a portion of the distal lumen 132A until a distal tip 112 of the needle 110 extends distally from the distal lumen opening 134A. The needle 110 can access the portion of the distal lumen 132A by way of a needle aperture 116 extending through a wall of the catheter 120 and communicating with the distal lumen 132A. As shown, the needle aperture (or needle access port) 116 can extend through a wall of the access section 122. Alternatively, the needle aperture 116 can extend through a wall of the dilation section 124 or the catheter section 126.

In an embodiment, the access guidewire 140 may inserted through the needle lumen 118 such that the access guidewire 140 is disposed within the access section 122. In some embodiments, access guidewire 140 may be configured to (i) stabilize the insertion site 90 once the needle 110 has been removed from the access section 122 and (ii) facilitate distal advancement of the access section 122. Similarly, in an embodiment, the advancement guidewire 142 may be inserted through a first extension leg 130A and into the distal lumen (or access lumen) 132A, where the advancement guidewire 142 is configured to facilitate distal advancement of the catheter section 126 along the vasculature.

Further details of catheter placement systems can be found, for example, in U.S. Pat. No. 10,376,675 and U.S. Published Applications 2019/0255294, 2021/0069471, 2021/0085927, 2021/0113809, 2021/0113810, 2021/0121661, 2021/0121667, 2021/0228843, 2021/0322729, 2021/0330941, 2021/0330942, 2021/0361915, 2021/0402153, 2021/0402149, 2022/0001138, each of which is incorporated by reference in its entirety into this application.

The system 100 further includes a skin nicking device (“device”) 150 coupled with the catheter 120. In some embodiments, the device 150 may be coupled with the dilation section 124 of the catheter 120. The device 150 includes a blade 154 coupled with a handle 152. The blade 154 extends distally away from the handle 152. In some embodiments, a proximal end of the blade 154 is supported by the handle 152. The handle 152 can include one or more gripping surfaces configured to enable a clinician to grasp the handle, e.g. between a thumb and forefinger, and to manipulate the skin nicking device 150. Optionally, the handle 152 can include one or more gripping features, such as ridges, ribs, or a second material such as silicone rubber or the like, and having a high friction coefficient. FIGS. 1-2 show the device 150 engaged with the catheter 120.

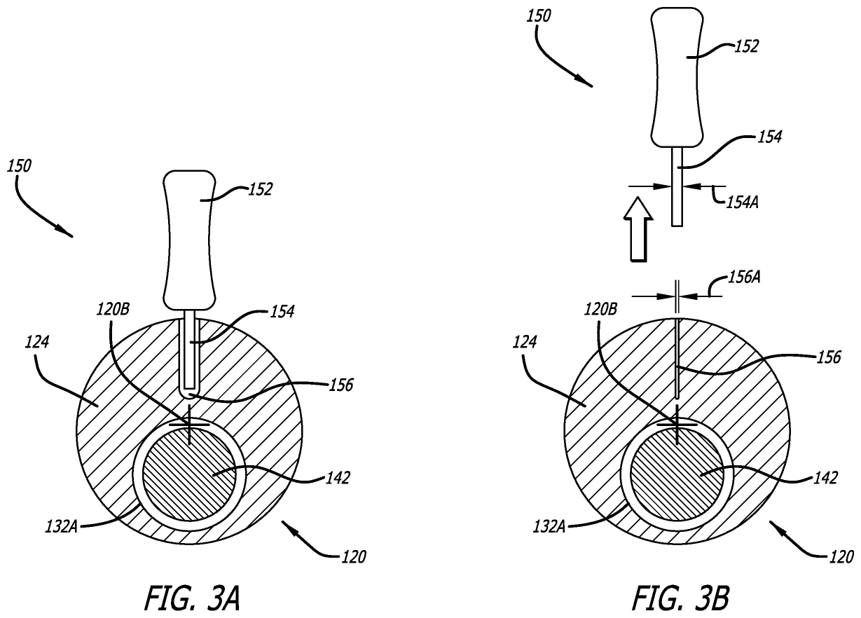

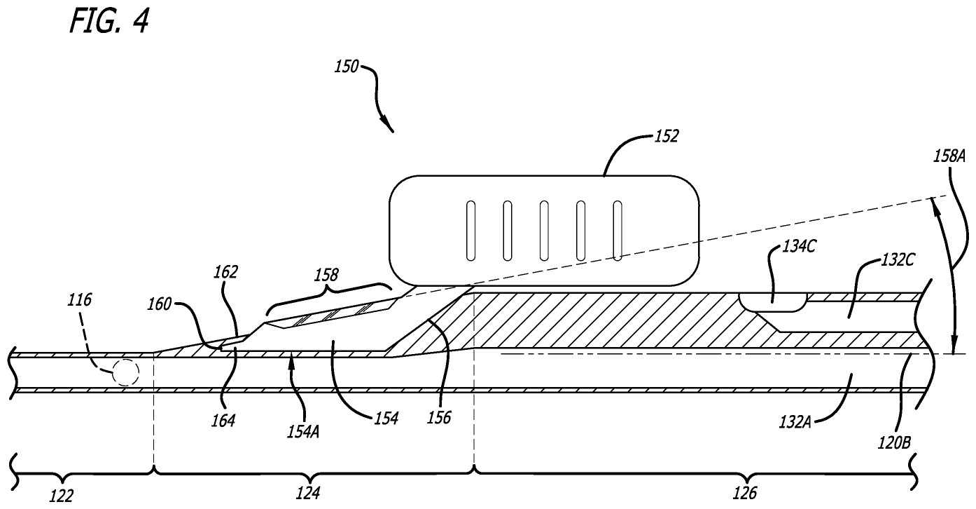

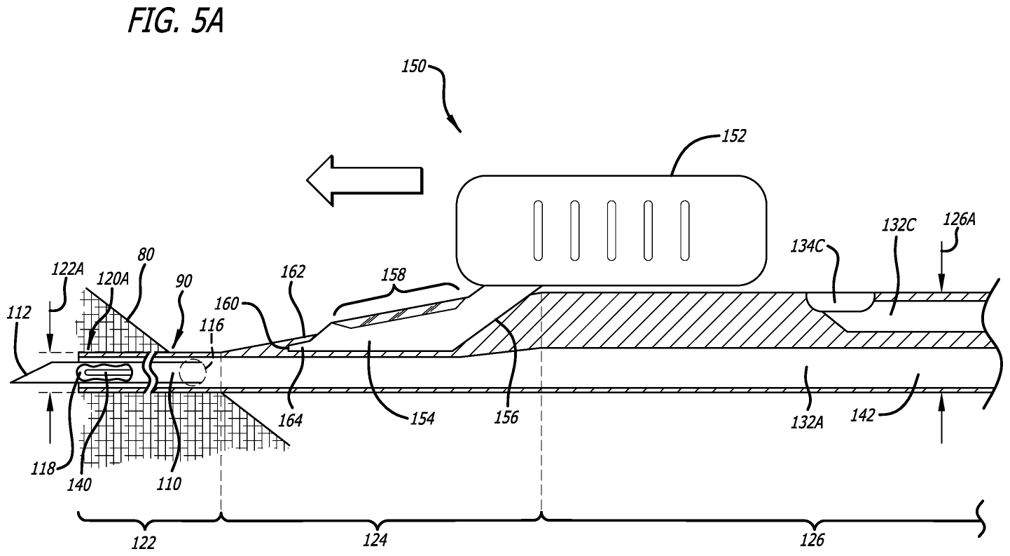

FIG. 3A shows a lateral cross-sectional view of the dilation section 124 of the catheter 120 with the device 150 engaged with the dilation section 124. FIG. 3B shows the lateral cross-sectional view of the dilation section 124 with the device 150 separated from the dilation section 124. FIG. 4 shows a longitudinal cross-sectional view of a portion of the system 100 with the device 150 engaged with the dilation section 124 of the catheter 120.

As shown in FIGS. 3A-3B, the catheter 120 includes a notch 156 extending radially inward from an outer surface of the catheter 120 (e.g., the dilation section 124) toward a longitudinal axis 120B (also see FIG. 4). The notch 156 extends longitudinally along the dilation section 124. In an embodiment, as shown in FIG. 3A, the notch 156 can be configured to receive at least a portion of the blade 154 of the device 150 therein. In some embodiments, the notch 156 defines a notch width 156A that, in a free state, is less than a thickness 154A of the blade 154. In an embodiment, the notch 156 can elastically deform to receive the blade 154 therein. As such, the notch 156 can engage the blade 154 in an interference fit so as to retain the blade 154 therein. In an embodiment, as shown in FIG. 3B, the notch 156 may be formed by a slit, where in the free state, i.e. in an elastically undeformed shape, a first longitudinal side wall can be in contact with a second longitudinal side wall opposite the first longitudinal side wall (i.e., the notch width 156A can be zero). As such, upon removal of the blade 154 from the notch 156, the notch 156 may transition to a closed state.

In an embodiment, as shown in FIG. 4, the blade 154 defines and inward edge 154A which may be flat and/or dull. As such, the inward edge 154A can mitigate cutting or damaging the catheter 120 when the blade 154 is disposed within the notch 156. The blade 154 further defines a sharp edge 158 configured to cut the skin of the patient during use. The sharp edge 158 can extend along a radially outward edge of the blade 154 extending radially away from the catheter 120. In an embodiment, the sharp edge 158 can protrude from the outer surface of the catheter 120, when the blade 154 is engaged with the notch 156. The sharp edge 158 defines an angle 158A with respect to the longitudinal axis 120B such that a distal portion of the sharp edge 158 is closer to the longitudinal axis 120B than a proximal portion of the sharp edge 158. In some embodiments, the angle 158A may be defined such that the sharp edge 158 is parallel with the outer surface of the dilation section 124. As shown in FIG. 4, the handle 152 may extend proximally along the catheter section 126. In some embodiments, the handle 152 may be disposed parallel with the longitudinal axis 120B.

In an embodiment, the notch 156 can further include a recess (or cavity) 160 disposed at a distal end of the notch 156, where the recess 160 defines a lip 162. The blade 154 can further include a tab 164 extending distally from a distal end of the blade 154. The tab 164 can extend longitudinally, laterally, or at an angle therebetween. The tab 164 can engage the recess 160 and the lip 162 when the blade 154 engages the notch 156. As such, the tab 164 when disposed within the recess 160 can retain the distal end of the blade 154 within the notch 156 so that the distal end of the blade 154 is disposed radially inward of the outer surface of the dilation section 124. Furthermore, the lip 162 and tab 164 structures may co-operate to prevent skin or tissues from passing between the blade 154 and the catheter 120 during insertion, forming a skin bridge, as described in more detail herein.

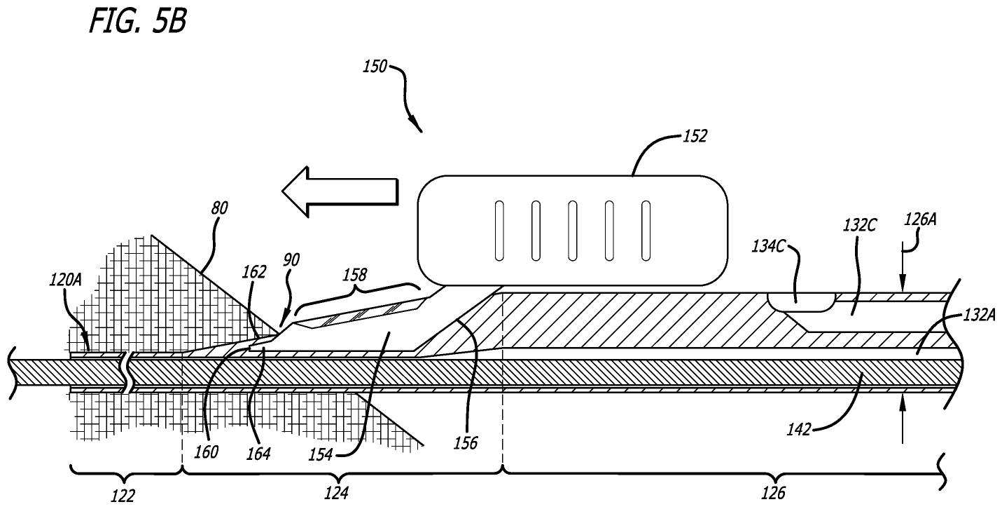

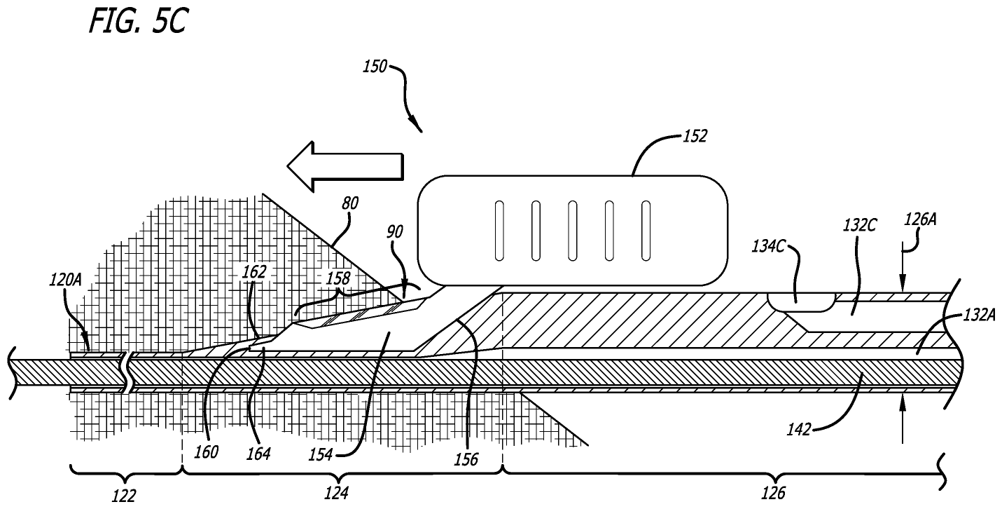

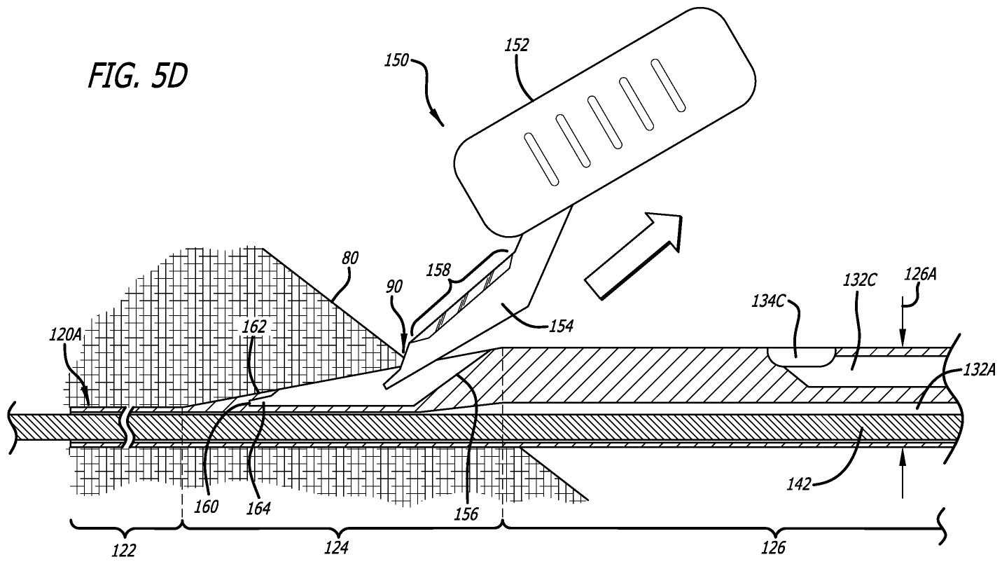

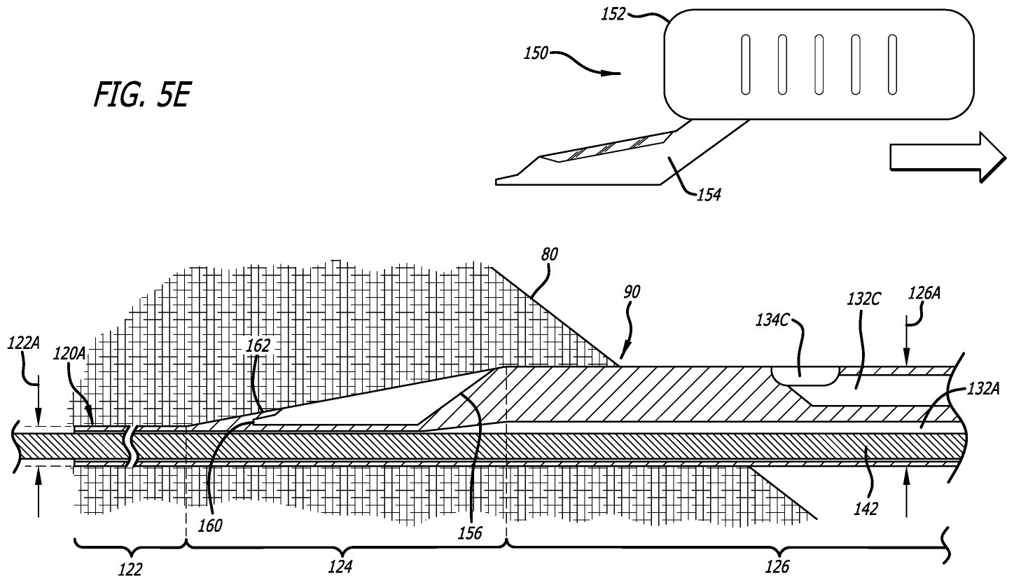

FIGS. 5A-5E illustrates cross-sectional views of the system 100 depicting various stages of an exemplary method of use, as described herein. As shown in FIG. 5A, the device 150 is engaged with the dilation section 124 of the catheter. A portion of the blade 154 is engaged with the notch 156 and retained therein via an interference fit. Further, the tab 164 extending from the distal end of the blade 154 is disposed within the recess 160 beneath the lip 162 to prevent the distal end of the sharp edge 158 from separating from the dilation section 124 as the catheter 120 is urged into the insertion site 90. Should the distal end of the sharp edge 158 separate from the catheter 120 during insertion, a portion of skin or tissue can pass between the blade 154 and the catheter 120 so as to form a skin bridge across the insertion site 90, thereby complicating the procedure. Shown is the needle 110 disposed within the distal lumen 132A along the access section 122 with the distal tip 112 of the needle 110 extending distally away from the distal end 120A. Shown also is the access guidewire 140 disposed within the needle lumen 118.

Initially, the needle 110 disposed within the access section 122 of the catheter 120 can puncture a skin surface 80 and access the vasculature of a patient. The blood flash indicator system (not shown) can draw a fluid proximally through a needle lumen 118 to confirm that the needle tip 112 is correctly placed within the vasculature by observation of a fluid color (e.g., red according to blood) and/or pulsatile flow. To note, the diameter 122A of the access section 122 is less than the diameter 126A of the catheter section 126. The diameter 122A of the access section 122 is such that should the needle 110 fail to correctly access the vasculature, the needle 110 and access section 122 can be withdrawn from the insertion site 90 and the insertion site 90 can be repaired without requiring vascular surgery.

In the instance where the correct vascular access has been confirmed, the access guidewire 140 can be advanced through the needle lumen 118 and into the vasculature. The needle 110 can then be removed proximally from the distal lumen 132A by way of the needle aperture 116 (see FIG. 1). The access section 122 can then be advanced over the access guidewire 140 until the dilation section 124 engages the insertion site 90 as shown in FIG. 5A. In an embodiment of the method, the access guidewire 140 can then be removed and the advancement guidewire 142 can be advanced through the distal lumen 132A and into the vasculature until a distal tip of the advancement guidewire 142 is placed at a target location within the vasculature.

As shown in FIG. 5B, as the dilation section 124 of the catheter 120 is urged into the insertion site 90, the tapered outer profile of the dilation section 124 can dilate the insertion site 90 from the first diameter 122A toward the second diameter 126A. In an embodiment of the method, a clinician can advance the catheter 120 by grasping the handle 152 and urging the handle distally. The blade 154 which is engaged with the notch 156, or more specifically, the tab 164 disposed within the recess 160, can engage the catheter 120 and urge the dilation section 124 into the insertion site 90. Advantageously, the blade 154 can provide additional columnar support to the dilation section 124 and can apply a distal force to the distal end of the dilation section 124, thereby pulling the proximal end of the dilation section 124 into the insertion site 90. This can mitigate kinking or collapse of the dilation section 124 as the dilation section 12 is urged into the insertion site 90. Further the tab 164 can enhance the engagement of the blade 154 with the dilation section 124 of the catheter 120. The lip 162 can direct skin tissues of the insertion site 90 over the radially outer edge of the blade 154 (i.e. sharp edge 158), preventing the skin tissues from passing between the blade 154 and the catheter 120 and forming a skin bridge.

Referring to FIG. 5C, the removable skin nicking device 150 is configured to facilitate dilation of the insertion site 90 to the second diameter 126A. As the dilation section 124 is urged into the insertion site 90, the sharp edge 158 of the blade 154, which is retained by the notch 156 and facing radially outward from the catheter 120, can engage an edge of the insertion site 90 to cut the skin tissues to facilitate dilation of the insertion site 90 to the second diameter 126A.

As shown in FIG. 5D, once the blade 154 has enlarged the insertion site 90 sufficiently, the clinician can remove blade 154 from the notch 156 of the catheter 120. The clinician can grasp the handle 152 and withdraw the device 150 proximally and upwards to disengage the blade 154 from the notch 156 and separate the blade 154 from the catheter 120. As noted, the material of the dilation section 124 can elastically deform to receive the blade 154 within the notch 156 and retain the blade 154 in an interference fit. In an embodiment, where the notch 156 is formed as a slit, the blade 154 can be removed from the notch 156 and the notch 156 can return to the undeformed position forming a substantially continuous circumferential surface. In an embodiment, where the notch 156 defines a gap, once the blade 154 has been removed from the notch 156, the radially inward pressure from the tissues surrounding the dilation section 124 can elastically deform the notch 156 to a closed state as shown in FIG. 3B and forming the substantially continuous circumferential surface.

As shown in FIG. 5E, the skin nicking device 150 can be removed and discarded in a “sharps” bin or similar suitable receptacle. The insertion site 90 is now dilated to accommodate the second diameter 126A and the clinician can continue to advance the catheter 120 over the advancement guidewire 142 until one or more of the distal lumen opening 134A, medial lumen opening 134B, or proximal lumen opening 134C are disposed at a target location within the vasculature. The advancement guidewire 142 can then be removed proximally from the distal lumen 132A.

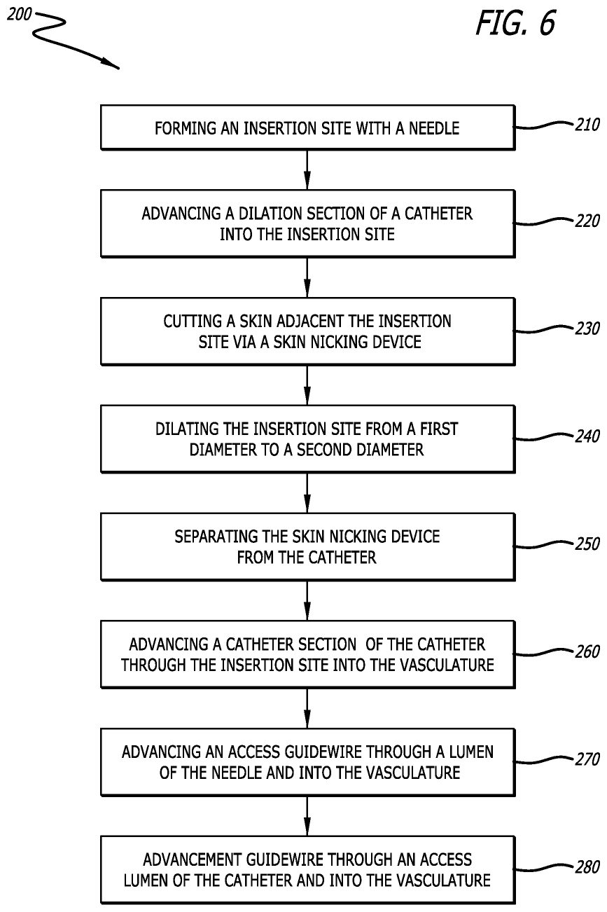

FIG. 6 illustrates a block diagram of method of placing the catheter within a vasculature of a patient that may include all or any subset of the following steps, actions, or processes. The method 200 may include forming an insertion site with the needle (block 210), where the needle is disposed within the access lumen of the access section of a catheter, and where the needle is inserted through the needle access port of the access section.

The method 200 may further include advancing the dilation section into the insertion site (block 220), where the dilation section is coupled with the skin nicking device. The method 200 may further include cutting a skin adjacent the insertion site (block 230), where the skin is cut with the sharp edge of the blade, and where cutting a skin may take place during advancement of the dilation section into the insertion site. As the blade includes the handle, advancing a dilation section may include applying a distally oriented force to the handle. As a portion of the blade is disposed within a notch, where the notch includes the cavity at the distal end thereof, and as the blade includes the tab extending distally away from a distal end of the blade, where the tab is disposed within the cavity, the distally oriented force applied to the handle may be transferred to the catheter via the tab disposed within the cavity.

The method 200 may further include dilating the insertion site from the first diameter of the access section to the second diameter of a catheter section (block 240). The method 200 may further include separating the skin nicking device from the catheter or more specifically the dilation section (block 250). The method 200 may further include advancing the catheter section through the insertion site into the vasculature (block 260).

The method 200 may further include advancing the access guidewire through the lumen of the needle and into the vasculature (block 270), and the method 200 may further include advancing the advancement guidewire through the access lumen and into the vasculature (block 280).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter placement system, comprising:
a catheter comprising:
a dilation section;
an access section extending distally away from the dilation section;
a catheter section extending proximally away the dilation section; and
a notch extending longitudinally along at least a portion of the dilation section; and
a skin nicking device coupled with the catheter, comprising:
a handle; and
a blade extending distally away from the handle, wherein a portion of the blade is disposed within the notch.

2. The system according to claim 1, wherein the catheter is a rapidly insertable central catheter.

3. The system according to claim 1, wherein the access section is mono-luminal and the catheter section is multi-luminal.

4. The system according to claim 1, wherein:
the access section defines a first outer diameter,
the catheter section defines a second outer diameter greater than the first outer diameter, and
the dilation section defines a tapered outer profile including:
the first outer diameter at a distal end of the dilation section; and
the second diameter at a proximal end of the dilation section.

5. The system according to claim 1, wherein the blade is removable from the notch.

6. The system according to claim 5, wherein the notch is formed of a slit such that:
upon removal of the blade from the notch, the notch transitions to a closed state, and
the outer surface of the dilation section forms a continuous circumferential surface.

7. The system according to claim 1, wherein the notch defines a notch width that is less than a thickness of the blade such that the notch defines an interference fit with the blade.

8. The system according to claim 1, wherein:
a dull edge of the blade is disposed within the notch, and
a sharp edge of the blade is directed radially outward from the catheter.

9. The system according to claim 8, wherein the sharp edge defines an angle with respect to a longitudinal axis of the catheter such that a distal portion of the sharp edge is closer to the longitudinal axis than a proximal portion of the sharp edge.

10. The system according to claim 9, wherein the notch is configured to align the blade with the longitudinal axis.

11. The system according to claim 8, wherein the sharp edge is parallel with an outer surface of the dilation section.

12. The system according to claim 1, wherein a distal end of the blade is disposed radially inward of the outer surface of the dilation section.

13. The system according to claim 1, wherein:
the blade includes a tab extending distally from the distal end of the blade,
the notch includes a cavity at a distal end of the notch, and
the tap is disposed within the cavity.

14. The system according to claim 13, wherein during use, a distally oriented force applied to the handle by a clinician is transferred to the catheter via the tab disposed within the cavity.

15. The system according to claim 1, wherein the handle extends proximally along the catheter section.

16. The system according to claim 1, wherein:
the catheter includes an access lumen extending along the access, dilation, and catheter sections, and
the access section includes a needle access port in fluid communication with the access lumen.

17. The system according to claim 16, further comprising a needle disposed within the access lumen between the needle access port and a distal end of the catheter.

18. The system according to claim 17, further comprising an access guidewire disposed within a lumen of the needle.

19. The system according to claim 16, further comprising an advancement guidewire disposed within the access lumen proximal access port.

20. A method of placing a catheter within a vasculature of a patient, comprising:
forming an insertion site with a needle disposed within an access lumen of an access section of a catheter, the needle inserted through a needle access port of the access section;
advancing a dilation section disposed proximal the access section into the insertion site, the dilation section coupled with a skin nicking device having a blade;
cutting a skin adjacent the insertion site with a sharp edge of the blade during advancement of the dilation section;
dilating the insertion site from a first diameter access section to a second diameter of a catheter section disposed proximal the dilation section during advancement of the dilation section;
separating the skin nicking device from the dilation section; and
advancing the catheter section through the insertion site into the vasculature.

21. The method according to claim 20, wherein:
the blade includes a handle, and
advancing a dilation section includes applying a distally oriented force to the handle.

22. The method according to claim 20, further comprising advancing an access guidewire through a lumen of the needle and into the vasculature.

23. The method according to claim 20, further comprising advancing an advancement guidewire through the access lumen and into the vasculature.

24. The method according to claim 20, wherein:
a portion of the blade is disposed within a notch extending along a least a portion of the dilation section, the notch including a cavity at a distal end thereof;
the blade includes a tab extending distally away from a distal end of the blade;
the tab is disposed within the cavity; and
the distally oriented force applied to the handle is transferred to the catheter via the tab disposed within the cavity.
